(19) United States
(12) Patent Application Publication
(10) Pub. No.: US 2016/0089077 A1
(43) Pub. Date: Mar. 31, 2016

(54) HYPERTENSION PREVENTION APP AND WEB BASED TOOLS

(71) Applicants: Christine Tinio Geronimo-Button, Manorville, NY (US); Terry Michael Button, Manorville, NY (US); Ashish Verma, Port Jefferson, NY (US)

(72) Inventors: Christine Tinio Geronimo-Button, Manorville, NY (US); Terry Michael Button, Manorville, NY (US); Ashish Verma, Port Jefferson, NY (US)

(21) Appl. No.: 14/868,315

(22) Filed: Sep. 28, 2015

Related U.S. Application Data
(60) Provisional application No. 62/055,918, filed on Sep. 26, 2014.

(51) Int. Cl. A61B 5/00; A61B 5/107; G06F 19/00; A61B 5/0205

(52) U.S. Cl. CPC A61B 5/486; A61B 5/742; A61B 5/7475; A61B 5/0205; A61B 5/107; A61B 5/4866; G06F 19/32; G06F 19/363; G06F 19/3481; A61B 2560/0475

(57) ABSTRACT

A computer system and method is disclosed as a support structure for patients with hypertension or prehypertension to encourage life style changes leading to dietary control, increase exercise and blood pressure control. The method includes automated messages and responses delivered in real time to encourage appropriate actions by the patient. A diary feature is provided for user input of various relevant parameters, such as foods consumed, exercise activities, body weight, blood pressure, and heart rate. The computer system and method may be implemented on a mobile computing device, such as a smart phone or wearable, a conventional personal computer, or a web service. Most functions will be provided on the mobile device, but some housekeeping and data visualization functions may be limited to the personal computer or web service implementations.

### CROSS-REFERENCE TO RELATED APPLICATION

This application claims priority to U.S. Patent Application No. 62/055,918, filed Sep. 26, 2014, the entire contents of which are incorporated by reference.

### FIELD OF THE INVENTION

This invention pertains to the area of software for the management of medical conditions, in particular, for the prevention and reduction of high blood pressure.

### BACKGROUND OF THE INVENTION

Hypertension is the primary cause of death of approximately 348,000 Americans each year. Approximately 76 million Americans, or 34% of the adult population, have hypertension and an additional 30 million have prehypertension. Hypertension or high blood pressure makes the heart work harder and the increased pressure can damage vessels and organs.

The risks from hypertension increase with age. Hypertension is more common in men, is more common in blacks and runs in families. Hypertension can result from inactivity, from using tobacco or alcohol, from too much salt, or from too little potassium or vitamin D in the diet. Hypertension can also result from stress. Hypertension is linked to high cholesterol, diabetes, and kidney disease.

Blood pressure is measured in millimeters of mercury (mm Hg). Systolic pressure is the higher number and represents the pressure while your heart is beating. Diastolic pressure is the lower number and represents the pressure while your heart is filling with blood. Normal blood pressure is 120/80 or less. High blood pressure is 140/90 or greater. Blood pressure between normal and high blood pressure (120-139)/(80-89) is termed prehypertension. People with prehypertension often become hypertensive within four years.

Hypertension makes the heart work harder resulting in damage to vessels and organ tissue. This increases their risk for ischemic heart disease (poor heart blood flow), stroke (damage to the brain), peripheral vascular disease (poor blood flow to the limbs), aneurysms (a balloon-like vascular structure that can burst), atherosclerosis (narrowing arteries), pulmonary embolism (blocks of vessels in the lung), cognitive impairment (brain damage) and chronic kidney disease.

Many life style changes have been identified that decrease blood pressure. The NIH Dietary Approach to Stop Hypertension (DASH) diet effectively reduces blood pressure by 11.4 mm Hg (systolic) and 5.5 mm Hg (diastolic). A DASH diet coupled with weight management and exercise reduces blood pressure by an average of 16.1 mm Hg (systolic) and 9.9 mm Hg (diastolic). Aerobic exercise alone also lowers blood pressure by 4 mm Hg (systolic) compared to controls. Changing to a low-sodium diet also reduces systolic pressure by 4.6 mm Hg. Reduced alcohol consumption lowers systolic pressure by 3.6 mm Hg and diastolic pressure by 1.8 mm Hg. Increased potassium reduces systolic blood pressure by 1.8 mm Hg and diastolic pressure by 1.0 mm Hg.

Many electronic tools are available on the web and as products that are geared toward weight loss, dietary control and/or exercise motivation. It is important to note that these tools, as a group, have failed. The number of hypertensives has rocketed due to poor eating and under activity to include 34% of all American adults, according to NIH data.

### SUMMARY OF THE INVENTION

A data-enabled apparatus and method is provided to assist persons with pre-hypertension or hypertension make life-style choices designed to reduce high blood pressure. The apparatus may be implemented on a handheld computer such as a smartphone or tablet, or it may be implemented on a laptop or desktop computer, or it may be implemented through a website.

The apparatus and method includes a software application, hereinafter termed a “Hypertension Prevention Module” (HPM) consisting of a computer application, running on computers such as Mac, Windows, iOS, Android, or as a web service. Coupled to the HPM is a “Records Management Database” (RMDB) that stores data entered by the user. The RMDB may also include pre-loaded data such as demographic and standardized medical data.

The HPM is programmed to interact with users in real time and process historical data entered by the user along with pre-loaded medical data to encourage proper eating, sufficient exercise, weight loss, reduced sodium consumption, increased consumption of potassium containing foods, and reduction of alcohol consumption. The apparatus and method is designed to be used by prehypertensive users to reduce their blood pressure normal values.

The HPM can be used as a method that routinely interacts with users over the course of a day. In various embodiments of the method, the following interactions may be employed:
Users may be asked questions that quiz their knowledge of lifestyle choices that affect blood pressure.
Users may be asked questions about their food choices at meal times.
Users may be asked about relevant medical data such as body weight and blood pressure.
Users may be given suggestions about what and when to eat.
Users may be given suggestions about their exercise.

Key components of the HPM may include:
A user interface provided in a mobile app, a laptop or desktop computer, or a website.
An educational component for an induction period of the first six weeks of the program using multiple choice questions.
A user database having data entry and review component (also termed herein a “diary”) that allows users to enter data such as medical data, food choices, and exercise that also allows users to track and review their progress.
A “Virtual Coach” component that displays intelligent messages and reminders to users in real time based on their data entry. For example, the Virtual Coach may remind the user to eat at a certain time, to eat certain foods, to exercise, or to record their medical data such as body weight and blood pressure. In addition, the Virtual Coach may provide daily and weekly summaries of the users' progress.
A mobile app that provides the educational, data entry, and Virtual Coach components, and provides messages and reminders to users in real time to instruct or encourage users to make desired choices likely to reduce hypertension.
A smart phone step counter that allows exercise analysis and automatic entry of exercise in the user diary.
Web based tools for use on Windows or Mac computers that allow virtually all features provided by the smart phone but includes additional features such as educational power points, questions and answers, reminders, schedule change and can generate an appropriate menu.

### BRIEF DESCRIPTION OF THE DRAWINGS

FIG. 1 is an overview of the parts of the HPM

FIG. 2 is an embodiment of a weekly summary shown on a smartphone.

FIG. 3 is a different embodiment of a weekly summary shown on a smartphone.

FIG. 4 is an embodiment of a daily summary shown on a smartphone.

FIG. 5 is flow chart of a user creation procedure.

FIG. 6 is flow chart of a user login procedure.

FIG. 7 is a flow chart of an embodiment of mobile app options.

FIG. 8 is a flow chart of an embodiment of web app or desktop app options.

FIG. 9 is a flow chart of an embodiment of Virtual Coach activities.

FIG. 10 is a flow chart of an embodiment of Virtual Coach activities at the end of the day.

FIG. 11 is a flow chart of an alternative embodiment of Virtual Coach activities at the end of the day.

FIG. 12 is a flow chart of an embodiment of Administrator options.

FIG. 13 is an embodiment of a weekly summary screen on a web app.

FIG. 14 is an alternative embodiment of a weekly summary screen web app, showing tabular and graphical blood pressure and heart rate data.

### DETAILED DESCRIPTION

### Hypertension Prevention Module Overview

As used herein, the term “computer” refers to any device having a microprocessor capable of running the software of the invention. The term may refer, for example, to a handheld device such as a smartphone or tablet computer, which may be termed a “mobile computer,” or a notebook. laptop computer, or desktop computer, which collectively may be termed a “personal computer.” In some embodiments, remote computers, of any design or configuration, may be employed. The terms “patient” and “user” are used interchangeably, unless the context indicates that a distinction is being made.

In an embodiment, the present invention provides a computer system for use by a user with hypertension or pre-hypertension, comprising a computer, a user interface operating on the computer, a records management database stored in a non-volatile electronic medium and operatively coupled to the computer, and a data management application running on the computer and coupled to the computer, and a display screen and keyboard for text input on the user interface; wherein the records management database comprises data relevant to hypertension, and the user enters relevant data into the records management database via the user interface; wherein the data management application is programmed to process data in the records management database and deliver pre-programmed messages to the user in real time; and wherein the data in the records management database comprises data relevant to hypertension and the messages and questions instruct or assist the user in real time to make lifestyle choices intended to reduce hypertension.

In another embodiment of the present invention, a computer-implemented method for reducing blood pressure in a patient with hypertension or prehypertension is provided, including providing a computer application having a user interface, wherein the computer application stores data in one or more databases in computer memory, providing a records management database stored in a non-volatile electronic medium operatively coupled to the computer, providing a data management application coupled to the user interface and running on a computer receiving patient data into one or more of the databases, wherein the data comprises medical parameters relevant to hypertension, querying the patient via the user interface on a routine basis regarding lifestyle factors that affect blood pressure, and receiving responses in real time from the patient that are stored in one or more of the databases, and providing automated suggestions on a routine basis and in real time to the patient on lifestyle choices, that if followed by the patient, are expected to reduce blood pressure in the patient.

In another embodiment of the present invention, a computer-implemented method is provided which uses a computer application running on a computer having a user interface, wherein the computer application stores data in one or more databases in non-volatile computer memory. The method receives patient data and stores the received data in a database, wherein the data comprises medical parameters from the patient relevant to hypertension. The method queries the patient via the user interface on a routine basis regarding lifestyle factors that affect blood pressure, and receives responses in real time from the patient, and storing the received responses in real time in a database. The method may further provide automated suggestions on a routine basis and in real time to the patient on lifestyle choices, that if followed by the patient, are expected to reduce blood pressure in the patient. The querying and providing responses are preferably performed on a routine basis, more preferably on a regular schedule. In preferred embodiments the application prompts the user on a regular schedule to enter patient data via the user interface. In alternative embodiments the application receives data automatically from sensors that are in communication with the computer.

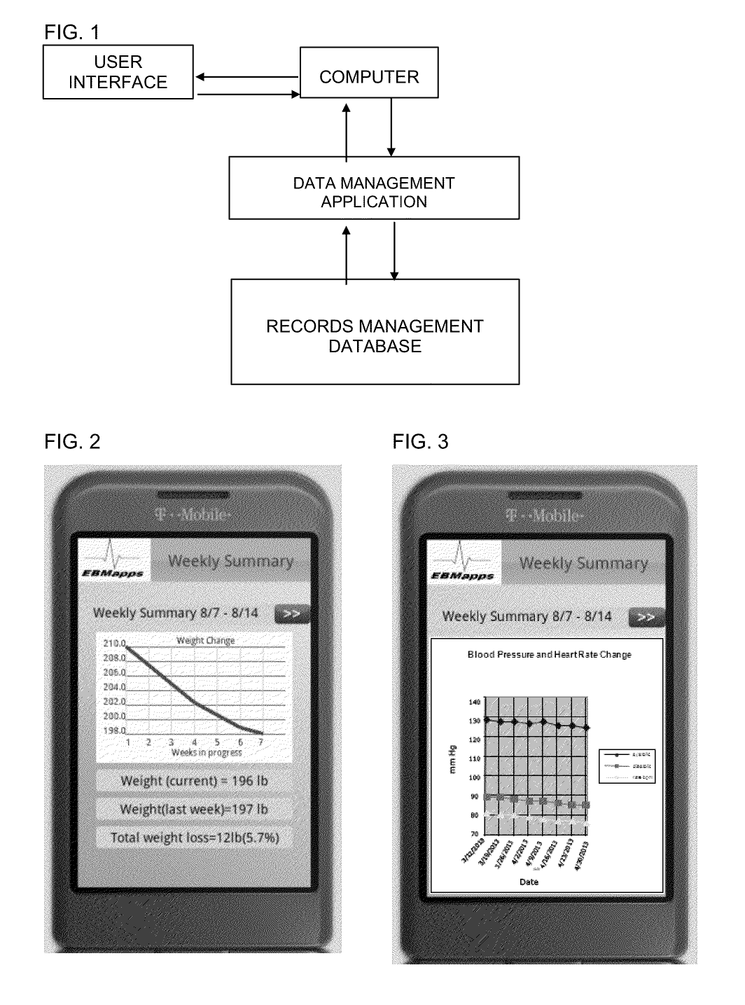

Referring to FIG. 1, a flow chart of an embodiment of the overall apparatus is illustrated. A user interface is provided, that typically will include a display screen and an input method such as an attached keyboard, which may be physical or a virtual keyboard displayed on a touch screen. The display screen may be, for example, a window in a computer application operating on a desktop or laptop computer, a smartphone screen, a tablet computer screen, or a web browser window.

The user interface may be controlled by a computer operatively linked to a Data Management Application (DMA), an application specially adapted for the instant purposes. The DMA may be a programmed application that runs on the computer, developed, for example, in C++, Ruby on Rails®, or Python™. In an embodiment, the DMA may be running on a remote server that accesses the computer through the internet or some other communication means.

In an embodiment, a Records Management Database (RMDB) is connected to the DMA. The RMDB is a database application designed to store data entered by the user, and preloaded data, such as demographic and standardized medical data, necessary for DMA activities.

Collectively, the entire apparatus comprising (in this embodiment) a user interface, computer, DMA, and RMDB, is termed the “Hypertension Prevention Module” (HPM).

In an embodiment, the DMA may communicate with other resources, such as servers connected to medical professionals, insurance companies, employers, personal trainers, and billing services. Typically, such communication will be wirelessly in the case of handheld devices, or through the internet in the case of desktop and laptop computers running the HPM. Wireless connectivity (e.g. via Wi-Fi) with a router which provides Internet communication is also contemplated for any type of device.

In an embodiment, the HPM may be implemented on several computer platforms, and users can employ any one of the platforms or a combination thereof. For example, the HPM may be implemented on a mobile platform, such as an iOS® or Android™ smartphone, a wearable computer (such as a smart watch or other jewelry), or a tablet computer. As used in this disclosure, the term “mobile app” refers to such a use.

The HPM may also be implemented on a desktop or laptop-type computer such as on computers running the Windows® or Macintosh® operating systems. Desktop or laptop-type computers are also referred to herein as “personal computers.” As used in this disclosure, the term “desktop app” refers to the HPM implemented on a desktop or laptop computer. The HPM may also be implemented on a website. As used in this disclosure, the term “website” refers to such a use.

In addition to the HPM, a user website may be provided, that will contain most or all of the tools in the users personal HPM, and the user will communicate with the HPM through the internet. The website may have additional tools not in the HPM. The website also may be configured to gather data, either personally identifiable or anonymized, for example for data aggregation purposes.

Data stored by mobile apps, desktop apps, and/or websites may be synchronized so as to permit a patient to run the RMDB and DMA on more than one platform. In other embodiments, the RMDB may run on a single platform while the DMA is run on multiple platforms.

In various embodiments, the HPM may be provided as a public service that anyone can enroll in through a public registration system. In other embodiments, the HPM may be provided as a closed system accessible only to members of a group, such as employees of a particular employer or health insurance members for a particular health insurance plan. For such groups, the HPM may be a medical benefit. In either embodiment, administrative supervision and/or clinical supervision (clinical coordinator) may be provided. For example, administrators or clinical coordinators may supervise the participation and progress of HPM participants (users).

For example, a clinical coordinator may contact a user who appears to have stopped using the system, for example, if a user hasn't entered data in several days. This can be an important leg of a motivational component of the HPM. The clinical coordinator can provide encouragement or help users deal with personal problems preventing them from fully participating in the program. Clinical coordinator may also manually flag unusual behavior (of the computer or users) for follow up. Clinical coordinators may also be notified automatically, or may manually flag, users who appear to be deteriorating on the program. For example a user whose blood pressure or blood sugar level increases steadily may need additional intervention beyond what the HPM can provide.

### Initiation Overview

New users of the HPM may be required to go through an initial registration step, including entering personal particulars and optionally billing information that will be stored in the RMDB.

Some of the information that may be required as part of the registration process includes:
Name and address, and other relevant identifying information, for example medical insurance number.
Credit card or other payment information (optional)
Sex
Date of birth
Initial blood pressure
Initial body weight
Initial height
Attesting that their physician has approved them for moderate stress exercise
Type of smart phone (or other mobile device)
Have a dietary target (as calculated below) of 1200 calories or more
Have a PC that has internet access and printing capabilities

Some general criteria that are envisioned are that each participant has a body mass index (BMI) over 27, that each participant be over 21, that any participant is not currently on medication to treat high blood pressure.

In addition, users may be required to provide the following information to initialize the module:
Times during the day that the Virtual Coach will remind them to eat well
times during the day for exercise and types of exercise or sports
times during the day the user will desire reminders to measure and record blood pressure and heart rate.

### Educational Tools

Some educational material for users is shown in the attachment. At user log-on the first time, the user may be directed to review the educational material before using other tools. The electronic educational material is composed of several sections:
Basic information about hypertension
Behavior modification
Controlling your diet
Exercise
Weight loss
Description of the module
Using your educational tools: smart phone

### Website Tools

In an embodiment, educational components may be available as separate selectable training modules on a website accessible to users of the system. For example, components covering Diet and Exercise, Using Web Tools and Using Smart Phone Tools may be available on the website.

While all tools are provided at the website, they may not be convenient for the user to employ in real time. To expect the user to log in to the website to enter diary information or to review their progress on a daily summary may be unrealistic. By contrast, advice from the Virtual Coach will ideally be provided in real time for optimum effectiveness. To address these issues, diary entries (adding data to the RMDB) and Virtual Coach messages can also be added or received through the user's smart phone or mobile device. In a preferred embodiment, the mobile app is designed to minimize the time required for diary input. An additional tool that may be provided on a mobile app is a step counter that provides full analysis of walking/jogging exercise including, distance traveled, speed (in mph) and calories burned.

### Operational Embodiments

In an embodiment, the HPM includes a “Virtual Coach,” which is an automated system driven by the DMA module of the HPM that automatically interprets and analyzes user activities based on rules pre-programmed into the HPM. For example, if the user enters food items that may be inappropriate, the Virtual Coach may send a message to the user recommending an alternative food choice. The Virtual Coach may also send reminders at specific times of the day, such as at times when exercise or eating is recommended. Such reminders may be via SMS text or as pop-up reminders on mobile devices. The Virtual Coach may send reminders for users to measure the blood pressure or body weight and enter it into their diary.

In an embodiment, the Virtual Coach may also ask the user about compliance with medications, including sending reminders to take medication when appropriate, and/or following up to confirm that that the user took the correct medication. The Virtual Coach may coordinate with a medical practitioner on this aspect, to adjust dosages or times when the user is reminded to take their medication.

In other embodiments of the Virtual Coach, a multiple choice question may be presented to users, for example early in the morning. The Virtual Coach may send a text message at each meal and snack reminding the user to eat appropriately and enter the foods eaten and salt consumed into their diary. For example, a Virtual Coach text message may be: “It is time for breakfast. Remember to eat healthy! Would you like to enter your meal into your diary? Don't forget to enter salt consumed.” The user may also receive a text message reminding them to exercise and to record their exercise in their diary. The Virtual Coach may provide the participant with a daily summary of their progress at the end of the day as well as a performance based notification encouraging them for the following day. Finally, the user may receive reminders to measure and record their body weight, their blood pressure and heart rate each week. Users may receive a weekly progress summary as well as a performance based encouragement notification for the coming week. The actual time the notifications are delivered are selectable by the user. These times can be modified by the user, for example, at the website.

In an embodiment, after the initiation process is completed, there is a preliminary phase to the program, in which each morning for the first six weeks just prior to breakfast, a multiple choice question will be sent to each user. In a preferred embodiment, the question will be sent via a mobile app, since most people are preparing for their day prior to breakfast and likely not sitting down at a desktop computer.

These multiple choice questions may do two things: (1) they may condition the user to think about controlling their eating and planning for exercise that day and (2) they may help reinforce the educational components of this program.

A sample multiple choice question may be:

Question: What is the best way to prevent hypertension?
A. A diet rich in fruits and vegetables
B. A low salt high potassium diet
C. Lose weight
D. All of the above

When the user selects the correct answer (here, the correct answer is “D”), the Virtual Coach will confirm that it is correct. When a user selects the wrong answer, the Virtual Coach will tell the user that the answer is wrong and provide the correct answer. The Virtual Coach will next ask the user to enter their breakfast foods into the diary.

Users may log in to use their electronic diary with the mobile application at any time. After logging on, users will be presented with a main menu of options for the HPM. In an embodiment, users may have options to enter dietary, exercise, weight, blood pressure or heart rate data into the HPM. They may also choose to monitor their exercise or look at performance summaries such as their daily or weekly summary.

### Mobile App Options

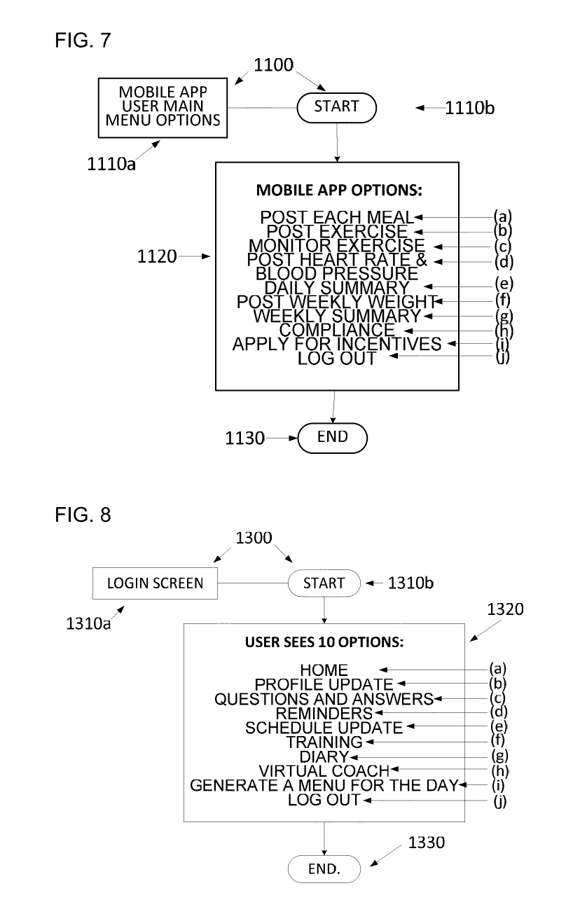

Various options are presented to the user on the mobile app that can be initiated by the user. An embodiment of a list of options is shown in FIG. 7.

In an embodiment, a “Post Each Meal” menu item on the main menu returns a set of button for meals to choose from, for example, breakfast, lunch, midafternoon snack, etc. After selecting the appropriate meal, the user will be able to enter foods eaten for that meal. If the user is responding to a text reminder to eat, this step may be omitted—the user may be taken directly to the meal input screen allowing them to enter servings for various food items. This screen lists all food groups. The user inserts a number to record the number of servings of each group. This is a very efficient method of recording foods consumed into a dietetic diary. An additional screen may also be presented listing common sources of dietary salt. The user can select the number of servings that were consumed at that particular meal. Upon submitting this data, salt intake will be approximated and stored in the user diary.

Another main menu item may be to enter exercise details. In an embodiment, various exercise activities may be presented, for example playing golf, walking, bicycling, playing tennis, etc. The user checks the type of exercise, enters the minutes of exercise completed and submits it.

In an embodiment, the HPM mobile app may include a step counter that that measures how many steps taken during running or jogging, when a smart phone or wearable is carried during these activities. Access to the step counter may be provided from the exercise option on the mobile app. The HPM mobile app may also include a timer, allowing users to press a button at the start of an activity and at the end. When the user selects stop exercise or it stops due to inactivity, a screen may appear showing distance walked, exercise time, average speed and calories burned. This information can be positive feedback to the user, and is automatically saved in the user's diary.

Other main menu items may be to enter body weight, blood pressure, and heart rate. The user may also be reminded to do these tasks at the times they have selected at in advance. Blood pressure and heart rate may optionally be acquired directly from wearable monitoring devices that are in communication with the computer.

When the user enters their weight to start a new week, a weekly summary may appear as shown in FIG. 2. This summary as shown compares user weight to the past week and provides a graph of weight over a several week period. It also provides total weight loss in pounds and as a percent of the user's initial body mass. The user target typically will be a weight loss of at least 3% over a 3 month period. This screen may also display an overview of user's overall performance in regard to controlling blood pressure. This screen (FIG. 3) graphically compares user blood pressure and heart rate to those over the past weeks. In this way improved blood pressure can be visually appreciated. This summary may also display minutes of exercise for the week compared to the user's target, calories consumed in exercise for the week, and average salt consumed for the week compared to the user's target.

Another option that may be provided are daily and weekly summaries of clinical targets, which include blood pressure and body weight, dietary entries, and exercise entries.

Another option that may be provided on the mobile app are for incentives. In this embodiment, health care plans can provide incentives to encourage use of the HPM. For example, users may receive gifts or discounts on purchases for participation in the HPM, and for meeting clinical targets, such as weight loss or blood pressure reduction, or for meeting dietary targets to reduce fat and sodium intake, increase potassium intake, or to log a regular program of moderate stress exercise.

If weight and/or blood pressure targets are met, the user may consent to the release of diary data (for the Diary Compliance, Weight Compliance, Blood Pressure (BP) Compliance) to an incentive program. In addition, a medical release may be emailed to the user to take to his/her physician to complete in order to validate weight loss and blood pressure reduction.

In addition to the user selectable menu items, the Virtual Coach may provide automated messages to the user at various times each day. For example, a text message from the Virtual Coach may be provided that encourages the user for the coming week based on their progress. In another embodiment, a user may be discouraged that their exercise isn't impacting their weight significantly. After all, 1200 calories typically burned for the week through exercise is only 0.3 lb lost. But exercise has other medical benefits. For example, users can be reminded that exercise significantly reduces blood pressure.

In various embodiments, the Virtual Coach may include notifications for a daily multiple choice question, a reminder to eat healthy at each meal, a reminder that it is time to exercise and a reminder that it's time to measure blood pressure and heart rate. In addition, the user may be asked to input diary dietary inputs, salt consumption input, exercise input and blood pressure/heart rate data. The Virtual Coach may send a daily summary and a performance based text of encouragement for the next day. Finally, on a weekly basis the user may be reminded to input their weight and after that input a weekly summary and performance based advice/encouragement for the next week is provided. While this may appear to be a cumbersome quantity of messages for the user (this could be as many as 136 tasks/interaction per week), the efficiency of the app is intended to make these interactions palatable for the user. The mobile app has been carefully designed and requires minimum effort to comply with requests for diary information. The tool constantly reminds and encourages but does not interfere with the users daily activities.

As described above, the Virtual Coach has many roles that are facilitated through the mobile app. In various embodiments, the Virtual Coach reminds users to eat well, exercise properly, and record their foods consumed, salt consumed, exercises completed, their weight and their blood pressure in the users diary. When data is not entered into the diary, the Virtual Coach may send several reminders.

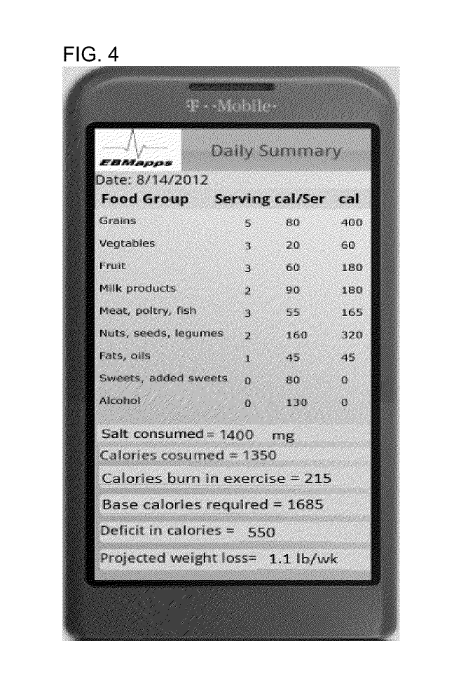

The Virtual Coach may also provide daily (FIG. 4) and weekly summaries (FIGS. 2 and 3), which are also selectable items on the main menu of the mobile app. The daily summary compares target food consumption with actual for the day. It also calculates the calories consumed and compares this to the calories used (base plus burned by exercise) to calculate the projected weekly weight loss or gain based on the days caloric consumption and expenditure. In addition, the sodium consumed is estimated and compared to the target value to show how well the user is complying with the program.

In an embodiment, the Virtual Coach may offer performance-based advice following a summary that will inform the user yet also be supportive. For example, if the user generally meets their food consumption target, the Virtual Coach will congratulate them on a great day. For minor excesses in one or two food categories or slightly exceeding the sodium intake target, the Virtual Coach may tell the user they had a good day and remind them to watch their food consumption relative to their targets. Should consumption result in a calculated weight gain, the Virtual Coach may remind the user that tomorrow is a new day and that they should focus on meeting their dietary targets. Whenever excess servings of fats are indicated, the Virtual Coach may remind the user that fats can increase blood fat level and so should be avoided. If thirty or more minutes of exercise is completed, the Virtual Coach may again congratulate them for a successful day. If exercise is completed but falls short of target, the Virtual Coach may encourage the user to have a better day tomorrow. If no exercise has been completed, the Virtual Coach may remind the user that tomorrow is a new day and they should focus on meeting their exercise target.

The weekly summary compares the users weight and blood pressure to the prior week. In addition the summary provides a plot of the users weight, blood pressure and heart rate for several past weeks while in the program. The summary also provides the total weight loss while in the study in pounds (lbs) and as a percent of total initial weight. The weekly summary also provides the total exercise minutes for the week compared to the target and the total calories burned through exercise for the week. Finally, the users blood pressure will be compared to determine if the user is still hypertensive or if blood pressure has returned to normal.

In an embodiment, the Virtual Coach will offer constructive performance based advice following each weekly summary depending on how well the user met their exercise and weight loss targets. Weight loss is a sensitive target with many variables in the near term. With diet and exercise as encouraged here, the long term trend should be towards lower body weight. It is important not to discourage a user that has a week of two where the actual weight does not meet expectations. Should the user lose a pound or more and meet their exercise target, they will be congratulated by the Virtual Coach. If weight loss is minimal but the exercise target is met or almost met, the Virtual Coach will encourage the user by telling them that sometimes weight loss is not closely correlated to exercise. Users that do not meet their weight loss or exercise goals will be reminded that exercise can be an important factor in reducing blood pressure and contributes to weight loss. To users that show no progress in weight loss over a few weeks but appear to meet dietary and exercise targets, the Virtual Coach may suggest that the user review the educational presentation regarding serving sizes or other remedial measures.

Health care savings can provide incentives to encourage use of the HPM. The HPM supports users in a process to lose weight, reduce fat and sodium intake, increase potassium intake, and follow a regular program of moderate stress exercise. Web and mobile tools may be designed to deliver information and receive data entered by the user. The HPM may also provide a summary of performance and offers performance based advice on a regular (daily and weekly) basis. To assist in the general goals of the HPM, a variety of monitors can be used to track user compliance and performance for the HPM. The monitors may include:
Did the user complete educational training?
What fraction of days did the user answer a multiple choice question?
How frequently was the multiple choice question answered correctly?
How frequently did the user input dietary information into their diary?
How frequently did the user input dietary sodium information into their diary?
How frequently did the user input exercise information into their diary?
How frequently did the user input blood pressure information into their diary?
How frequently did the user input weight information into their diary?
How frequently did the user's recorded dietary information meet target values?
How frequently did the user's recorded dietary sodium information meet target values?
How frequently did the user's recorded exercise information meet target values?
How frequently did the user's recorded blood pressure information meet target values?
How frequently did the user's recorded weight meet that calculated based on dietary and exercise information?
Validated weight compliance (comparison of entered weight loss compared to weight loss obtained from the user's physician)
Validated blood pressure reduced to normal (user's physician certification that user blood pressure is in the normal range)

In an embodiment, the HPM can be used with a wellness program, for example from a health insurance provider or employer, that provides incentives to diary compliance, weight compliance, blood pressure (BP) compliance, validated weight loss and validated blood pressure reduction. From diary inputs these data can be easily tracked and made available to the user. The user is always able to see their compliance with important module monitors and track their progress.

In any such incentive program, it is envisioned that users must consent to have their personal information transmitted to the program. Thus, in an embodiment, the user may be asked to consent to the release of diary data (for the diary compliance, weight compliance, blood pressure (BP) Compliance) to the wellness program. In addition, a medical release may be sent to the user to take to his/her physician to complete and return to validate weight loss and blood pressure reduction.

### Web Based Tools

In addition to the mobile app (diary and Virtual Coach), in an embodiment, a website may be provided giving users access to web-based tools through a standard internet browser. These tools may include most of those found in the mobile app, for example, a meal diary, salt consumption diary, exercise diary, weight diary, blood pressure/heart rate diary, daily summary and weekly summary. These tools will allow users to enter diary data and receive reminders and messages through the website as an alternative, or in addition to, the mobile app. In addition, the website may provide additional tools for user profile update, questions and answers, study reminders, user schedule update, dynamic training, generate a dietary menu, detailed account information, and a method to opt out of the study. Details of the web based tools are provided below.

For example, questions and answers may be made available to the user to help them understand hypertension and the process that they are following to prevent it. At a “question and answer” screen, the user may search a hypertension-related database of frequently asked questions. If a user has a question not in the database, a messaging system may be provided allowing the user to ask a clinical coordinator the question. The clinical coordinator may optionally add such new questions, and the answer, to the question and answer database.

Another website feature may be a reminder screen that notifies users of missing diary information, such as a missing meal or missing exercise data.

Another website feature may be a “schedule update” screen, where the user can change their meal time or exercise text reminders. This feature provides adaptability in the system.

Another website feature may be a “Training” option, where the user can view content, for example, a Power Point-type slide presentation, optionally with audio, on topics such Diet and Exercise, Using Web Tools, or Using Mobile App Tools. An example of educational material that might be presented in the Training option is shown in the attachment.

Another website feature may be a Diary option that allows a review and input of meals, sodium (salt), exercise, weight blood pressure and heart rate. This tool provides a flexible option for users to add to omitted entries, review previous entries, and change previous entries. This window also provides daily food targets and historical food data.

For example, the Diary option may have a screen for the sodium (salt) diary. This screen may list many common high salt dietary items. Users can input the number of servings of any of these items at each meal. An estimated salt intake is calculated based on a base salt intake (800 mg/day) plus the salt consumed in any of these items selected. The daily target may be adjusted but is typically less than 1500 mg/day.

The Diary option may also have an exercise diary screen, allowing users to enter and edit exercise data. Exercise may be entered by the user each day for each exercise type (for example, walking, cycling, or swimming). A summary of the user's most recently entered exercise is also posted in a table and graph format as shown.

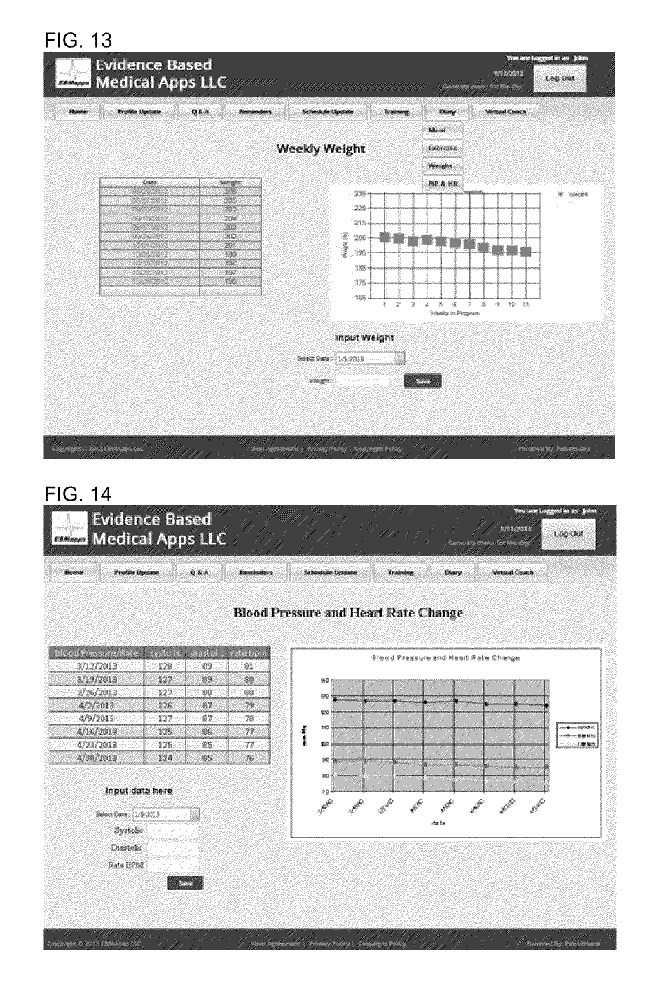

The Diary option may also have a screen showing body weight (FIG. 13), showing the users weight they have entered each week. An option may be provided on this page to enter body weight, as an alternative to the mobile app. Historical weight data may be given in tabular and graphical formats as illustrated in FIG. 13. This graphic depiction of weight (ideally trending downward in most cases) is expected to make a large impact on users in terms of understanding progress and their weight loss goal, and as a motivational tool. Users may modify their exercise list at the schedule update screen.

The Diary option may also have a screen showing blood pressure and heart rate data entered by users on a daily or weekly basis (FIG. 14). An option may be provided on this page for users to enter their blood pressure and heart rate each week, as an alternative to using the mobile app. Historical blood pressure and heart rate data may be displayed in the tabular and graphical formats as shown in FIG. 14. A graphic depiction of blood pressure (ideally trending downward in most cases) can make a large impact on the user in terms of understanding progress and their blood pressure goal, and as a motivational tool.

Various summaries of relevant data may be provided under the diary tab. For example, FIGS. 13 and 14 show summaries of body weight and blood pressure presented in tabular and graphical formats. Options may be provided to show various time ranges for summary data, for example, over periods of days, weeks, or months.

A Virtual Coach option may be provided, that includes various drop down menus that may show daily and weekly summaries of parameters and projections provided by the HPM. For example, the daily summary may show an overview of calories consumed, calories used, projected weight loss, minutes of exercise, sodium intake and blood pressure for each day of the most recent week. The user may also be able to review historical daily summary data. Similarly, a weekly summary may be provided, showing an overview of calories consumed, calories used, projected weight loss, minutes of exercise, sodium intake and blood pressure for recent weeks. The daily and weekly summaries may include graphical display options, allowing a user to view, for example, their blood pressure over the past eight weeks in a graphical view. This can be a powerful tool to help users understand their progress. This can also be a strong motivational tool, showing that the various lifestyle choices and changes recommended by the HPM and Virtual Coach result in desired body changes such as lower body weight and lower blood pressure.

A “Menu of the Day” option on the main webpage menu may also be provided, giving users a suggested menu for meals and snacks for that day. These generated meal plans may help users get started on planning to eat right and are expected to be an important leg of the overall program. In an embodiment, these meal menus may be created by a random combination of different sets of breakfast, morning snack, lunch, afternoon snack, dinner and evening snacks. Each daily menu includes the number of servings for each meal and is designed to meet the nutritional targets for that user, such as total calories, protein, sodium, etc. Many additional features can be added to the menu including creation of shopping list and shopping carts to satisfy the foods on the menu.

### Calculators

In an embodiment, a number of calculators may be provided on the website and in the mobile and computer applications. Inputs for these calculations may come from the questionnaire and the user diary. Example calculations may include:

Body Mass Index (BMI)
Inputs: weight (lb) and height (inches)

BMI=[weight/(height²)]×703

### Distance Walked and Speed:

Inputs: gender, steps taken (cell phone step counter), walking time (cell phone step counter in minutes)
Stride factor=0.413 for females and 0.415 for males

Distance walked (miles)={[stride factor×height (inches)/12]×steps taken}/5,280

Speed (mph)=[distance walked (miles)/walking time (min)]/60

Calories Burned in Walking:
Inputs: weight (lb), miles walked (computed above)

Calories burned=weight×miles walked×0.3

### Calories Consumed:

Inputs: servings of grain, vegetables, fruit, milk, meat, nuts, fats and sweets (from user diary)

Calories consumed=grain servings×80+vegetable servings×25+fruit servings×180+milk servings×90+meat servings×55+nut servings×125+fat servings×45+sweet servings×80.

Calories Required Per Day:
Inputs from questionnaire: age, weight, height, gender and activity level (1-4)
correction for gender is add 5 for males and subtract 161 for females
correction for activity level is multiply by 1 for sedentary, multiply by 1.2 for low
activity, multiply by 1.27 for active and multiply by 1.45 for very active.

Calories required per day=[9.9×weight (kg)+2.25×height (cm)+4.92×age in years+gender correction]×activity correction

Projected Weight Loss Per Week: (from One Day's Data)
Inputs from calculators: calories required per day, calories burned, calories consumed

Projected weight loss=7×(calories required+calories burned−calories consumed)/3500

### User Security

Authentication or proper qualification and/or identification of users may be assured by a secure user login method. All data collected from users and transmitted over computer networks may be stored and transmitted in encrypted formats compliant with HIPAA standards.

### App Flow Charts

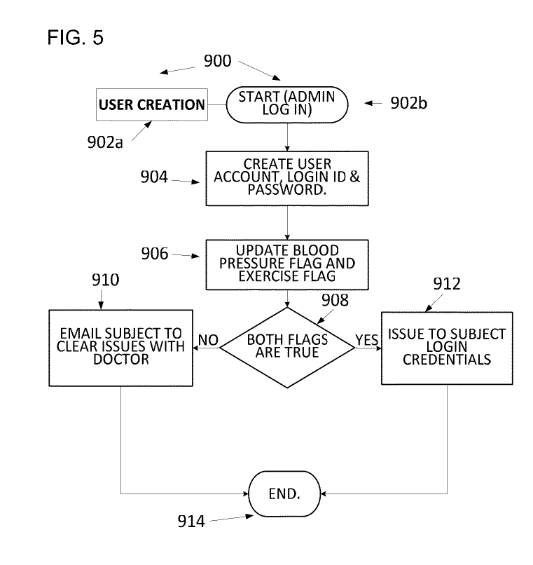

Various flow charts illustrating an embodiment of the operational features of the inventions are shown in FIGS. 5 to 12.

FIG. 5 is a flow chart of a user creation procedure. Users are prompted to create a log in ID and password at step 904. In this example, users are required to enter initial blood pressure and exercise data at step 906. Step 906 also sets flags if the data is sufficient for participation in the study. The flags are evaluated in step 908. For example, a severely obese patient may require additional supervision, so step 908 would initiate contact with the users physician. If the user meets the parameters for the program, they will be issued login credentials and can begin using the HPM.

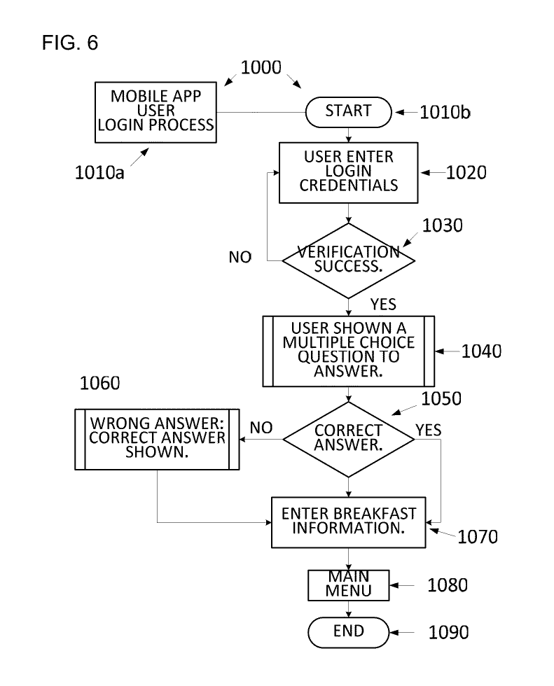

FIG. 6 shows a typical user login procedure. After verification success step 1030, the user may be presented with a multiple choice question providing a simple thought provoking and educational component.

FIG. 7 is a flow chart of main menu options for the mobile app. FIG. 8 is a log in screen for the desktop or web app, showing menu items that might be used in the HPM.

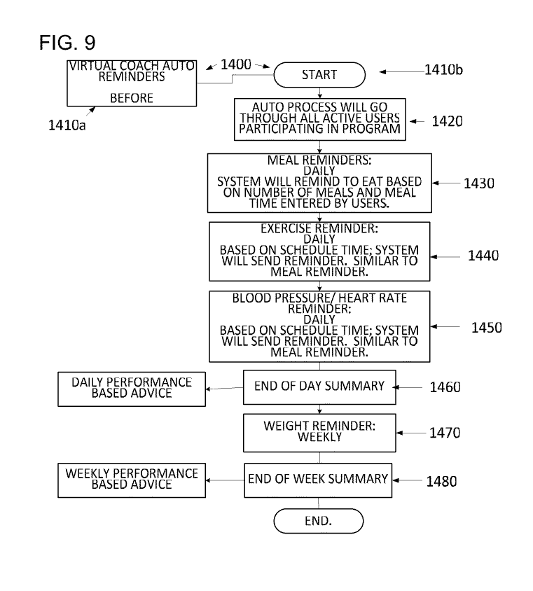

FIG. 9 is a flow chart of typical activities of the Virtual Coach. In many cases, the beginning (step 1400b) may be initiated automatically such as with an automated reminder at a certain time of day (step 1410a). Various activities that the Virtual Coach might use are shown in steps 1430, 1440, and 1450.

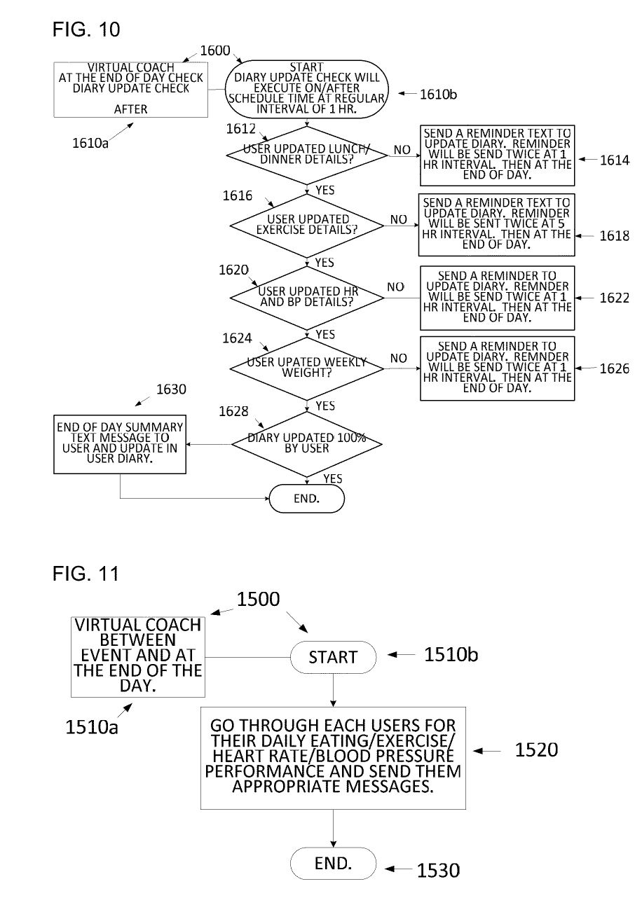

FIG. 10 illustrates Virtual Coach activities at the end of the day, such as shortly before bedtime. The Virtual Coach will review expected user inputs, such as food entries (step 1612) and exercise details (step 1616), and send the user a reminder (steps 1614 and 1618 respectively) if the data entry is absent or appears to be mistaken or inaccurate. For example, a user who enters ten servings of broccoli for dinner may be presumed to have made an error, so the Virtual Coach may ask for confirmation or correction.

FIG. 11 shows the Virtual Coach review of data at the end of the day, such as shortly before bedtime, and sends users appropriate messages.

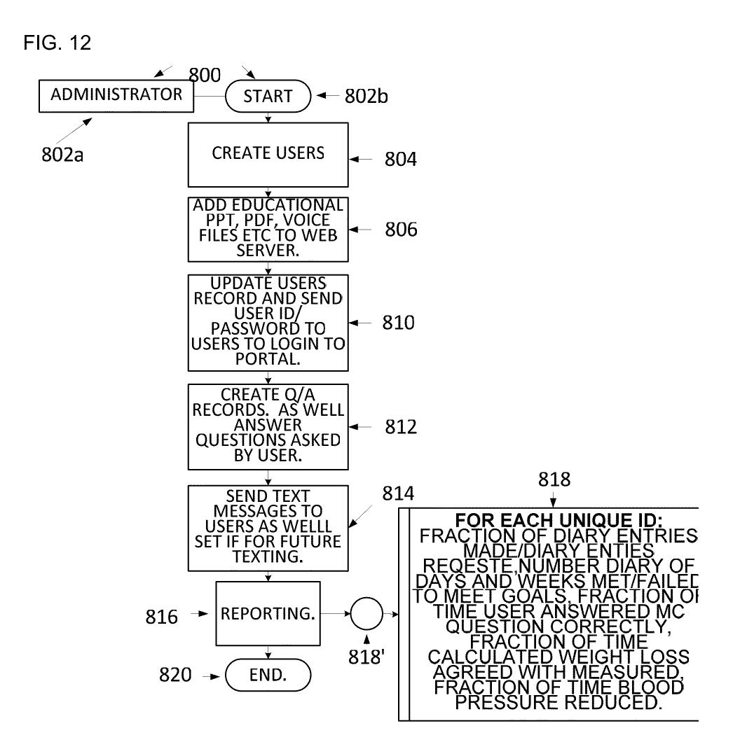

FIG. 12 is a flow chart of administrator actions that may be provided in some embodiments of the HPM. These may be actions by clinical administrators or computer administrators. For example, user records may be updated by the administrator (step 810) if a user is having computer trouble.

### REFERENCES

1. Appel L J, T J Moore, E Obarzanek, et al. For the DASH Collaborative Research Group (1997) A clinical trial of the effects of dietary patterns on blood pressure. N Engl J Med 336:1117-24.
2. Smith P J, J A Blumenthal, M A Babyak, L Craighead, K A Welsh-Bohmer, J N Browndyke, T A Strauman, and A Sherwood (2010) Effects of the dietary approaches to stop hypertension diet, exercise, and caloric restriction on neurocognition in overweight adults with high blood pressure. Hypertension 55:1331-8.
3. Whelton S P, A Chin, X Xin, and J He (2002) Effect of aerobic exercise on blood pressure: a meta-analysis of randomized, controlled trials. Ann Intern Med 136:493-503.
4. Whelton P K, L J Appel, M A Espeland, et al. For the TONE Collaborative Research Group (1998) Sodium reduction and weight loss in the treatment of hypertension in older persons: a randomized controlled trial of nonpharmacologic interventions in the elderly (TONE). JAMA 279:839-46.
5. Xin X, J He, M G Frontini, L G Ogden, O I Motsamai, and P K Whelton (2001) Effects of alcohol reduction on blood pressure: a meta-analysis of randomized controlled trials. Hypertension 38:1112-7.
6. Whelton P K, J He, J A Cutler, F L Brancati, L J Appel, D Follmann, and M J Klag (1997) Effects of oral potassium on blood pressure. Meta-analysis of randomized controlled clinical trials. JAMA 277:1624-32.
7. Your Guide to Lowering Your Blood Pressure With DASH http://www.nhlbi.nih.gov/health/public/heart/hbp/dash/new_dash.pdf

1. A computer system for use by a user with hypertension or pre-hypertension, comprising
a. a computer;
b. a user interface operating on the computer, the user interface having a display screen and keyboard for text input;
c. a records management database stored in a non-volatile electronic medium operatively coupled to the computer;
d. a data management application coupled to the user interface and running on the computer;
wherein the records management database comprises data relevant to hypertension, and the user enters relevant data into the records management database via the user interface;
wherein the data management application is programmed to process data in the records management database and deliver pre-programmed messages to the patient in real time; and
wherein the messages and questions instruct or assist the user in real time to make lifestyle choices intended to reduce hypertension.

2. The system of claim 1, wherein the data relevant to hypertension comprises medical data about the patient, selected from blood pressure measurements, heart rate measurements, body weight measurements, body dimension measurements, exercise quantity, potassium intake, and salt intake, and combinations thereof.

3. The system of claim 1, wherein the data relevant to hypertension comprises dietary information of foods consumed by the user.

4. The system of claim 1, wherein the user interface operates on a platform selected from a website, a smartphone, a tablet computer, a desktop or laptop computer, and a wearable computer.

5. A computer-implemented method for reducing blood pressure in a patient with hypertension or prehypertension comprising
a. providing a computer application having a user interface, wherein the computer application stores data in one or more databases in computer memory;
b. providing a records management database stored in a non-volatile electronic medium operatively coupled to the computer;
c. providing a data management application coupled to the user interface and running on a computer;
d. receiving patient data into one or more of the databases, wherein the data comprises medical parameters relevant to hypertension;
e. querying the patient via the user interface on a routine basis regarding lifestyle factors that affect blood pressure, and receiving responses in real time from the patient that are stored in one or more of the databases; and
f. providing automated suggestions on a routine basis and in real time to the patient on lifestyle choices, that if followed by the patient, are expected to reduce blood pressure in the patient.

6. The method of claim 5, wherein the computer application is an application running on a mobile device, a desktop or laptop-type computer, or a website.

7. The method of claim 5, wherein the queries via the user interface on a routine basis are provided to the patient at least three times per day.

8. The method of claim 5, wherein the queries via the user interface on a routine basis are provided to the patient at least five times per day.

9. The method of claim 5, wherein the automated suggestions provided on a routine basis are provided at least three times per day.

10. The method of claim 5, wherein the automated suggestions provided on a routine basis are provided at least five times per day.

11. The method of claim 5, further comprising providing a summary of the patient's historic medical data for review by the patient.

12. The summary of claim 11, wherein the historic medical data comprises body weight and/or blood pressure data.

13. The method of claim 5, wherein a clinical supervisor monitors the progress of the patient.

14. The method of claim 5, further comprising querying the records management database to obtain a question relating to hypertension, wherein the question presented is based on data entered by the patient into the records management database.

15. The method of claim 5, further comprising presenting summary data to the patient at regular intervals, wherein the data relates to factors related to hypertension, and optionally include data entered by the patient into the RMDB.

16. The method of claim 5, wherein a set of multiple-choice questions is presented periodically to the patient via the user interface, wherein the questions are directed to lifestyle choices that affect hypertension, and wherein the patient is prompted to answer each multiple-choice question, and the answers are stored in the records management database.
